(19) United States
(12) Patent Application Publication
(10) Pub. No.: US 2017/0281349 A1
(43) Pub. Date: Oct. 5, 2017

(54) OSTEOSYNTHETIC IMPLANT AND MANUFACTURING METHOD THEREOF

(71) Applicant: OLYMPUS CORPORATION, Tokyo (JP)

(72) Inventors: Takamitsu SAKAMOTO, Tokyo (JP); Hirofumi TANIGUCHI, Tokyo (JP); Masato TAMAI, Tokyo (JP)

(73) Assignee: OLYMPUS CORPORATION, Tokyo (JP)

(21) Appl. No.: 15/629,290

(22) Filed: Jun. 21, 2017

Related U.S. Application Data
(63) Continuation of application No. PCT/JP2014/084403, filed on Dec. 25, 2014.

(51) Int. Cl. A61F 2/28; A61L 27/30; A61L 27/56; A61F 2/30; A61L 27/04

(52) U.S. Cl. CPC A61F 2/28; A61F 2/30767; A61L 27/047; A61L 27/56; A61L 27/306; A61F 2002/3092; A61F 2002/30838; A61F 2002/30971; A61F 2310/00425; A61F 2002/30003; A61L 2430/02; A61L 2420/02

(57) ABSTRACT

For the purpose of firmly fusing a low-cost osteosynthetic implant having high osteoconductivity with a bone in a short period of time after implanting without having to perform treatment to restore surface hydrophilicity, a osteosynthetic implant is provided with a substrate that is formed of magnesium or a magnesium alloy and a porous anodic oxide coating that is formed on a surface of the substrate, wherein the anodic oxide coating has an outer surface that, due to the sizes and distribution of pores that are formed when generating the anodic oxide coating by means of anodic oxidation treatment, structurally prevents water from entering the pores while maintaining the hydrophilicity thereof.

### CROSS-REFERENCE TO RELATED APPLICATIONS

This is a continuation of International Application PCT/JP2014/084403, with an international filing date of Dec. 25, 2014, which is hereby incorporated by reference herein in its entirety.

This application claims the benefit of International Application PCT/JP2014/084403, filed on Dec. 25, 2014, the content of which is incorporated herein by reference.

### TECHNICAL FIELD

The present invention relates to an osteosynthetic implant and a manufacturing method thereof.

### BACKGROUND ART

In the related art, there is a known biodegradable implant material in which corrosion resistance in a biological subject is increased by forming a porous coating on a magnesium-alloy substrate (for example, see Patent Literature 1). Osteoconductivity is one of functions required for an osteosynthetic implant. Osteoconductivity is related to hydrophilicity at the surface of the osteosynthetic implant, and it is known that the osteoconductivity is decreased when the hydrophilicity is decreased. Thus, proposed means for restoring the bone compatibility of a surface by enhancing the hydrophilicity include physical methods, such as sandblasting, and chemical methods, such as etching by means of acid or the like (for example, see Patent Literature 2).

### CITATION LIST

### Patent Literature

{PTL 1} PCT International Publication No. WO 2013/070669
{PTL 2} Publication of Japanese Patent No. 5186376

### SUMMARY OF INVENTION

### Technical Problem

An object of the present invention is to provide a low-cost osteosynthetic implant having high osteoconductivity that can be firmly fused with a bone in a short period of time after being implanted without having to perform treatment to restore surface hydrophilicity, and to provide a manufacturing method thereof.

### Solution to Problem

An aspect of the present invention is an osteosynthetic implant including: a substrate that is formed of magnesium or a magnesium alloy; and a porous anodic oxide coating that is formed on a surface of the substrate, wherein the anodic oxide coating has an outer surface that, due to the sizes and distribution of pores that are formed when generating the anodic oxide coating by means of anodic oxidation treatment, structurally prevents water from entering the pores while maintaining hydrophilicity thereof.

In the above-described aspect, the outer surface of the anodic oxide coating may have a surface structure in which the Cassie-Baxter model is dominant over the Wenzel model.

In the above-described aspect, at the outer surface of the anodic oxide coating, a ratio of areas of openings of the pores and areas of portions other than those may be equal to or less than 1.8.

In the above-described aspect, at the outer surface of the anodic oxide coating, a ratio of areas of openings of the pores and areas of portions other than those may be equal to or less than 1.

In the above-described aspect, a coating thickness of the anodic oxide coating may be 1 to 5 μm, and an average pore size of the pores opened in the outer surface may be equal to or less than 5 μm.

In the above-described aspect, a coating thickness of the anodic oxide coating may be 1 to 5 μm, and an average pore size of the pores opened in the outer surface may be equal to or less than 1 μm. By doing so, it is possible to more reliably prevent droplets from entering the pores even if there is variability in the coating thickness.

In the above-described aspect, a macro-scale surface roughness of the outer surface of the anodic oxide coating may be equal to or less than 1 μm.

In the above-described aspect, the anodic oxide coating may be formed by immersing the substrate formed of magnesium or a magnesium alloy in an electrolyte, which contains phosphoric acid at 0.1 mol/L or less, which contains ammonia or ammonium ion at 0.2 mol/L, which does not contain fluorine and chlorine, and which has a pH of 9-13, and electricity is passed therethrough.

Another aspect of the present invention is an osteosynthetic-implant manufacturing method in which anodic oxidation treatment is applied, in which a substrate formed of magnesium or a magnesium alloy is immersed in an electrolyte, which contains phosphoric acid at 0.1 mol/L or less, whici contains ammonia or ammonium ion at 0.2 mol/L, which does not contain fluorine and chlorine, and which has a pH of 9-13, and electricity is passed therethrough.

### BRIEF DESCRIPTION OF DRAWINGS

FIG. 1 is a longitudinal cross-sectional view showing a surface portion of an osteosynthetic implant according to an embodiment of the present invention.

FIG. 2A is a schematic diagram showing a Wenzel model for explaining hydrophilicity.

FIG. 2B is a schematic diagram showing a Cassie-Baxter model for explaining hydrophilicity.

FIG. 3 is a graph showing the relationship between the carbon mass concentration at a surface and osteoconductivity.

FIG. 4 is a graph showing the relationship between the elapsed time after implanting and the bone-fusing rate.

FIG. 5A is a diagram showing an electron micrograph of an anodic-oxide-coated surface in a First Example of the osteosynthetic implant in FIG. 1.

FIG. 5B is a diagram showing an electron micrograph of a tomographic image of FIG. 3A.

FIG. 6A is a diagram showing an electron micrograph of an anodic-oxide-coated surface in a Second Example of the osteosynthetic implant in FIG. 1.

FIG. 6B is a diagram showing an electron micrograph of a tomographic image of FIG. 4A.

FIG. 7A is a diagram showing an electron micrograph of an anodic-oxide coated surface (non-carbonized portion) in a Comparative Example of the osteosynthetic implant.

FIG. 7B is a diagram showing an electron micrograph of a tomographic image of FIG. 5A.

FIG. 7C is a diagram showing an electron micrograph of an anodic-oxide coated surface (carbonized portion) in FIG. 5A.

### DESCRIPTION OF EMBODIMENT

An osteosynthetic implant 1 according to an embodiment of the present invention will be described below with reference to the drawings.

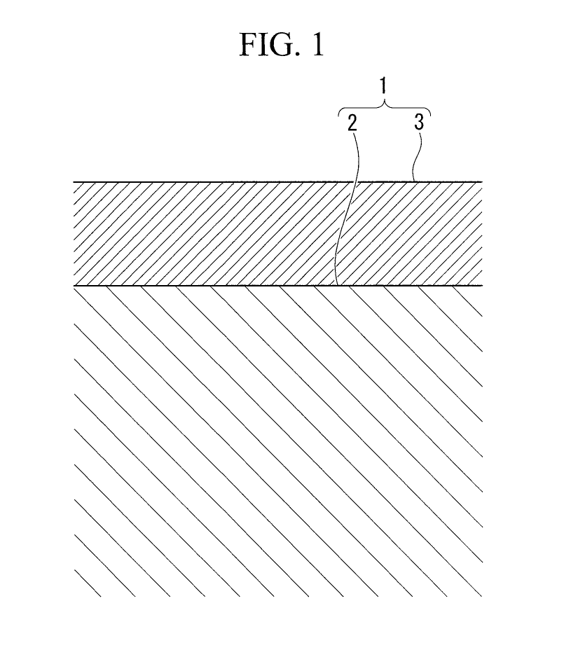

As shown in FIG. 1, the osteosynthetic implant 1 according to this embodiment is provided with a substrate 2 formed of magnesium or a magnesium alloy, and a porous anodic oxide coating 3 that is formed on a surface of the substrate 2.

The anodic oxide coating 3 has an outer surface that, due to the sizes and distribution of pores 3 formed when generating the anodic oxide coating 3 by means of anodic oxidation treatment, structurally prevents water (hereinafter, referred to as droplets) W from entering the pores 3a while maintaining the hydrophilicity.

Specifically, the macro-scale structure of the anodic oxide coating 3 generated by the anodic oxidation treatment is made smooth. In other words, the anodic oxide coating 3 has an outer surface in which the macro-scale roughness is suppressed to be equal to or less than 1 μm.

The macro-scale roughness refers to geometric shapes that have frequencies that are lower than those of the pores associated with the anodic oxidation, and that have frequencies that are higher than the geometric deviation of an article to be subjected to anodic oxidation.

The anodic oxide coating 3 has an outer-surface surface structure in which, while maintaining the hydrophilicity, adsorption of moisture in the pores 3a is decreased by controlling the micro-scale structure of the anodic oxide coating 3 generated by the anodic oxidation treatment. In other words, the ratio of the areas of openings of the pores 3a, which are opened in the outer surface of the anodic oxide coating 3, and the areas of portions other than those is set so as to be equal to or less than 1.81.

The operation of the thus-configured osteosynthetic implant 1 according to this embodiment will be described below.

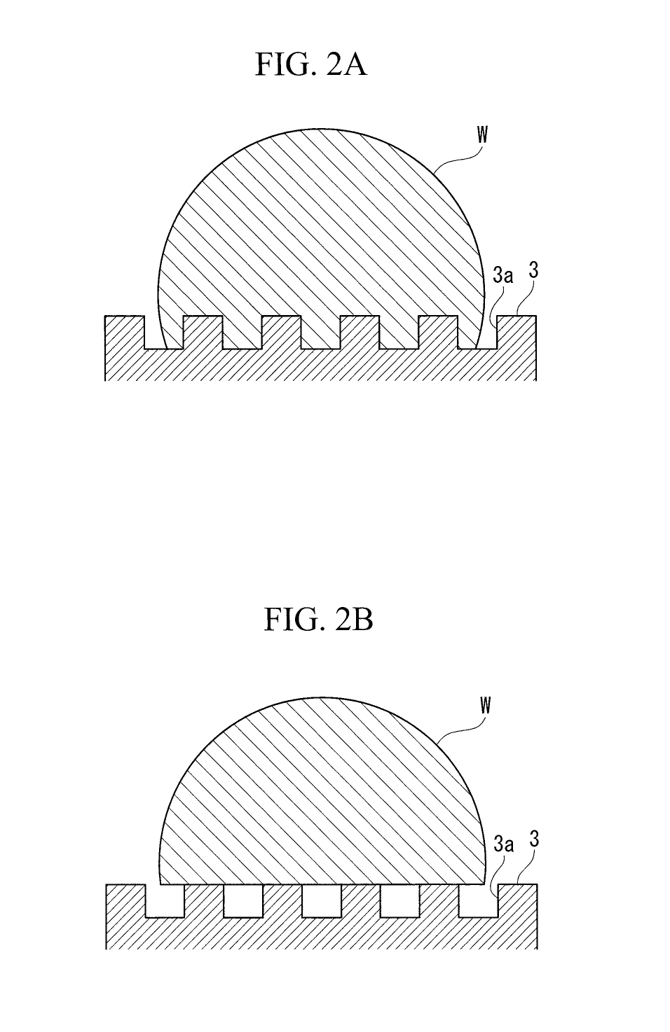

With the outer surface of the anodic oxide coating 3 of the osteosynthetic implant 1 according to this embodiment, because the ratio of the areas of the openings of the pores 3a opened in the outer surface and the areas of the portions other than those is set so as to be equal to or less than 1.8, the Cassie-Baxter model shown in FIG. 2B becomes more dominant than a so-called Wenzel model shown in FIG. 2A.

Therefore, as shown in FIG. 2B, this achieves a state in which a high hydrophilicity is achieved because the rough outer surface and the liquid surface appear to be in contact with each other over a large area due to a large degree of micro-scale irregularities caused by the pores 3a in the surface, whereas the droplets W and the outer surface are in point contact with each other due to the presence of the numerous pores 3a which the droplets W cannot enter.

Thus, because it is difficult for moisture to enter the pores 3a in the outer surface of the osteosynthetic implant 1, it is difficult for carbon in the air to be taken into the osteosynthetic implant 1 during the storing period until being implanted into a biological subject, and thus, it is possible to prevent carbide from being generated due to bonding of moisture and carbon.

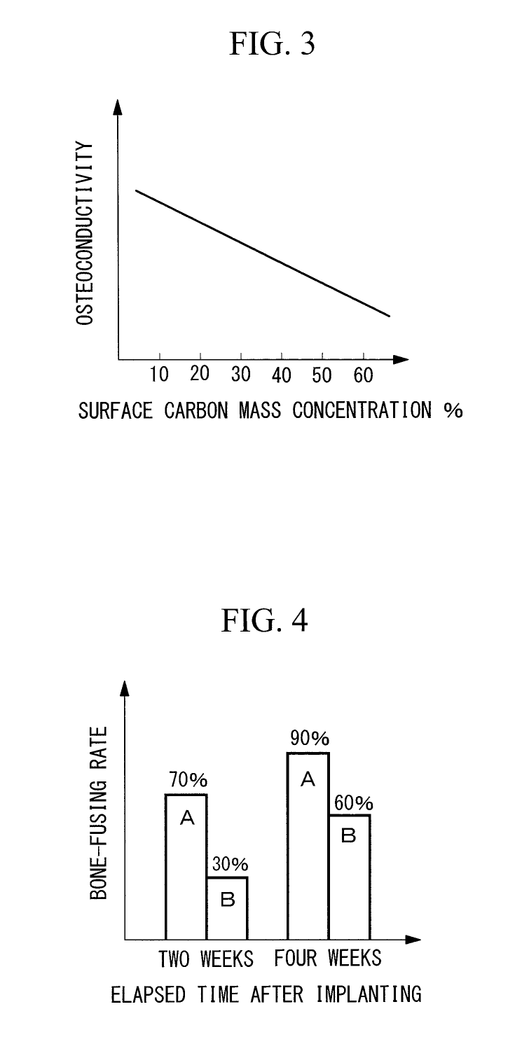

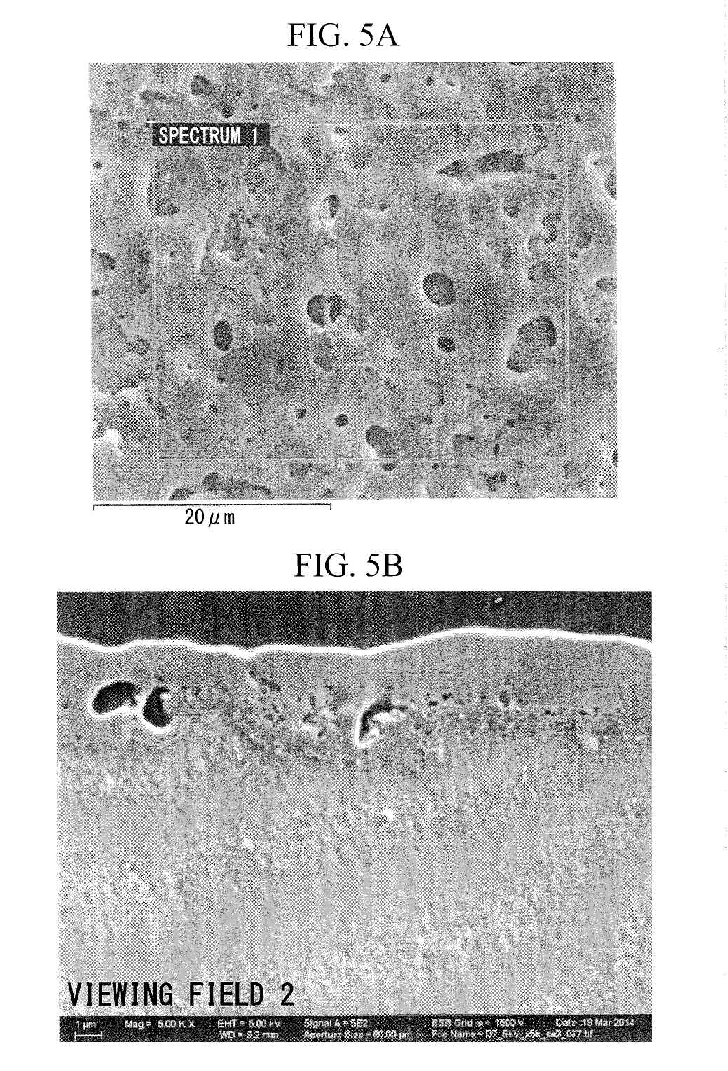

As shown in FIG. 3, there is a relationship between the carbon mass concentration at the surface of the osteosynthetic implant 1 and the osteoconductivity such that the osteoconductivity is decreased with an increase in the carbon mass concentration. As shown in FIG. 4, with pure titanium (sample A) that has a carbon mass concentration of 17% and that has been subjected to etching treatment, the bone-fusing rates in the case of implantation into a rat were 70% two weeks after implanting and 90% four weeks after implanting. With pure titanium (sample B) that has a carbon mass concentration of 64%, the bone-fusing rates were 30% after implanting and 60% four weeks after implanting.

In the orthopedic field, in general, fixtures are removed and rehabilitation is started after performing load relief for a certain amount of time. For example, the targets for load relief are three weeks for the antebrachial bone, four weeks for the clavicle, and three to five weeks for a rotator-cuff tear. If the fusion rate of the osteosynthetic implant 1 and bone is improved, it is possible to start rehabilitation early, specifically, it is desirable that the bone-fusing rate at the point in time three weeks after implantation in a rat be 90%. On the basis of the relationships in FIGS. 3 and 4, the carbon mass concentration with which the bone-fusing rate reaches 90% three weeks after implanting is determined to be approximately 6%.

Specifically, because the bone-fusing rates of the pure titanium that has the carbon mass concentration of 17% in the case of implantation in a rat are 70% after two weeks and 90% after four weeks, by interpolation, the bone-fusing rate three weeks after implanting is 80%. In addition, because the bone-fusing rates of the pure titanium that has the carbon mass concentration of 64% in the case of implantation in a rat are 30% after two weeks and 60% after four weeks, by interpolation, the bone-fusing rate three weeks after implanting is 45%. Accordingly, the proportional relationship between the bone-fusing rate at the point in time three weeks after implanting and the carbon mass concentration is expressed by Expression (1) below:

Y=−0.76X+94 (1),

where Y is the bone-fusing rate at the point in time three weeks after implanting, and X is the carbon mass concentration.

On the basis of the above Expression (1), in the case in which the bone-fusing rate at the point in time three weeks after implanting is 90%, the carbon mass concentration is approximately 5.26%, in other words, equal to or less than 6%. Therefore, in the case in which the carbon mass concentration at the surface is equal to or less than 6%, it is possible to maintain such a surface osteoconductivity that allows rehabilitation to be started early.

When the osteosynthetic implant 1 according to this embodiment is implanted into bone tissue, the outer surface of the anodic oxide coating 3 comes into contact with body fluid, and thus, biodegradation thereof is started. As has been described above, there is an advantage in that, because the osteosynthetic implant 1 according to this embodiment possesses a high hydrophilicity due to prevention of carbide generation at the surface thereof, the osteosynthetic implant 1 possesses a high osteoconductivity, thus fusing early and firmly with bone tissue in the surrounding area thereof. Subsequently, during the period until the anodic oxide coating 3 and the substrate 2 are eliminated due to biodegradation, the osteosynthetic implant 1 maintains mechanical strength, and thus, it is possible to stably complete healing of the bone tissue in the surrounding area.

Although this embodiment is structurally configured so that the Cassie-Baxter model becomes more dominant than the Wenzel model by setting the ratio of the areas of the openings of the pores 3a opened in the outer surface and the areas of the portions other than those is equal to or less than 1.81, it is preferable that the ratio be equal to or less than 1.

Conditions for the Wenzel model and the Cassie-Baxter model to coexist include that the angles at the openings of the pores 3a in the outer surface be smaller than the droplet contact angle. The contact angle of the magnesium anodic oxide coating 3 is about 30°.

Therefore, the conditions are satisfied when the thickness of the anodic oxide coating 3 is 1 to 5 μm, preferably 2 to 5 μm, and the opening size of the pores 3a is equal to or less than 5 μm, preferably equal to or less than 1 μm, which makes it possible to achieve coexistence of the Wenzel model and the Cassie-Baxter model, and thus, it is possible to make it difficult for moisture to enter the pores 3a. It is possible to more reliably prevent droplets from entering the pores 3a even if there is variability in the coating thickness.

Next, a manufacturing method of the osteosynthetic implant 1 according to this embodiment will be described.

In order to manufacture the osteosynthetic implant 1 according to this embodiment, anodic oxidation is applied, in which a magnesium alloy is immersed in an electrolyte, which contains phosphoric acid or phosphate at 0.0001 to 5 mol/L, preferably, 0.1 mol/L or less, which contains ammonia or ammonium ion at 0.01 to 5 mol/L, preferably, 0.2 mol/L, which does not contain fluorine and chlorine, and which has a pH of 9 to 13, and electricity is passed therethrough.

It is preferable that the electrolyte temperature when passing the electricity be controlled to 5 to 50° C. Before applying the anodic oxidation, it is preferable that the substrate 2 be treated by being immersed in acidic and alkaline solutions. Doing so makes it possible to dissolve and remove a natural oxide coating on the magnesium or magnesium alloy surface and impurities thereon such as processing oil, a releasing agent, or the like used during shape processing, and thus, the quality of the anodic oxidation coating is enhanced. Using immersion in an acidic solution and an alkaline solution in combination is more preferable because doing so makes it possible to dissolve and remove insoluble impurities that are formed when immersed in one of the solutions by means of immersion in the other solution. It is possible to use a solution such hydrochloric acid, sulfuric acid, phosphoric acid, or the like as the acidic solution, and it is possible to use a solution such as sodium hydroxide, potassium hydroxide, or the like as the alkaline solution. Regarding the temperatures of the respective solutions used in the immersing treatment, although the effects thereof are exhibited even when kept at room temperature, greater impurity dissolving and removal effects are expected when immersion is performed in a state in which the temperatures are kept at 40 to 80° C.

The anodic oxidation treatment is performed by using the substrate 2 immersed in the electrolyte as the anode, and by connecting a power source between the substrate 2 and a cathode material that is similarly immersed.

There is no particular limitation to the power source to be used, although it is possible to use a DC power source or an AC power source, it is preferable to use a DC power source.

In the case in which a DC power source is used, it is preferable to use a constant-current power source. There is no particular limitation to the cathode material, for example, it is possible to suitably use a stainless-steel material or the like. It is preferable that the surface area of the cathode be greater than the surface area of the substrate 2 to be subjected to the anodic oxidation treatment.

In the case in which a constant-current power source is employed as the power source, the current density at the surface of the substrate 2 is equal to or greater than 20 A/dm². The electricity-passing time is 10 to 1000 seconds. When passing electricity by using the constant-current power source, although the applied voltage is low when the passing of electricity is started, the applied voltage increases with the passage of time. The voltage of the applied voltage that is finally reached when stopping the passing of electricity is equal to or greater than 350 V.

By doing so, it is possible to manufacture the osteosynthetic implant 1 having the anodic oxide coating 3 with the above-described structure by means of a single-step anodic oxidation treatment.

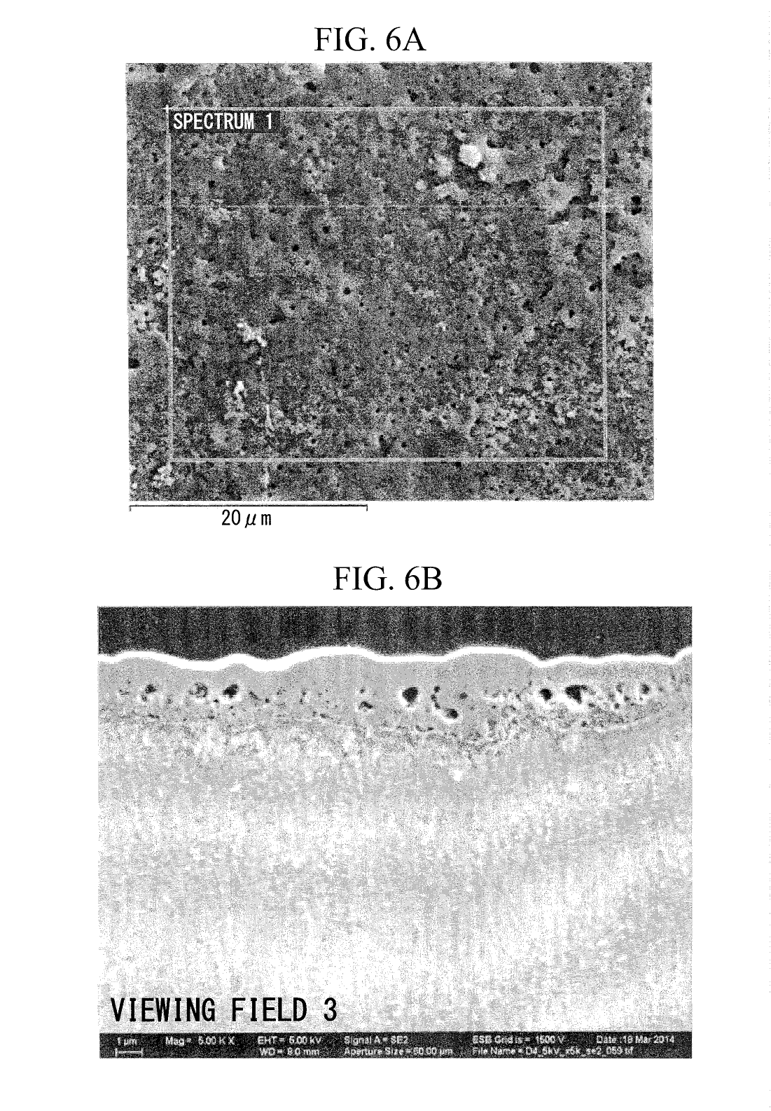

FIG. 5A shows an electron micrograph of an outer surface of a osteosynthetic implant 1 that is manufactured by means of a First Example of the manufacturing method according to this embodiment, and FIG. 5B shows a micrograph of a tomographic image showing a portion from the anodic oxide coating 3 to the substrate 2.

In the First Example, manufacturing is performed by setting the phosphoric-acid concentration to 0.05 mol/L, the current density at the surface of the substrate 2 to 20 A/dm², and the voltage of the applied voltage that is finally reached when stopping the passing of electricity to 400 V.

By doing so, the mass concentration of carbon atom at the outer surface of the anodic oxide coating 3 was 5.05%.

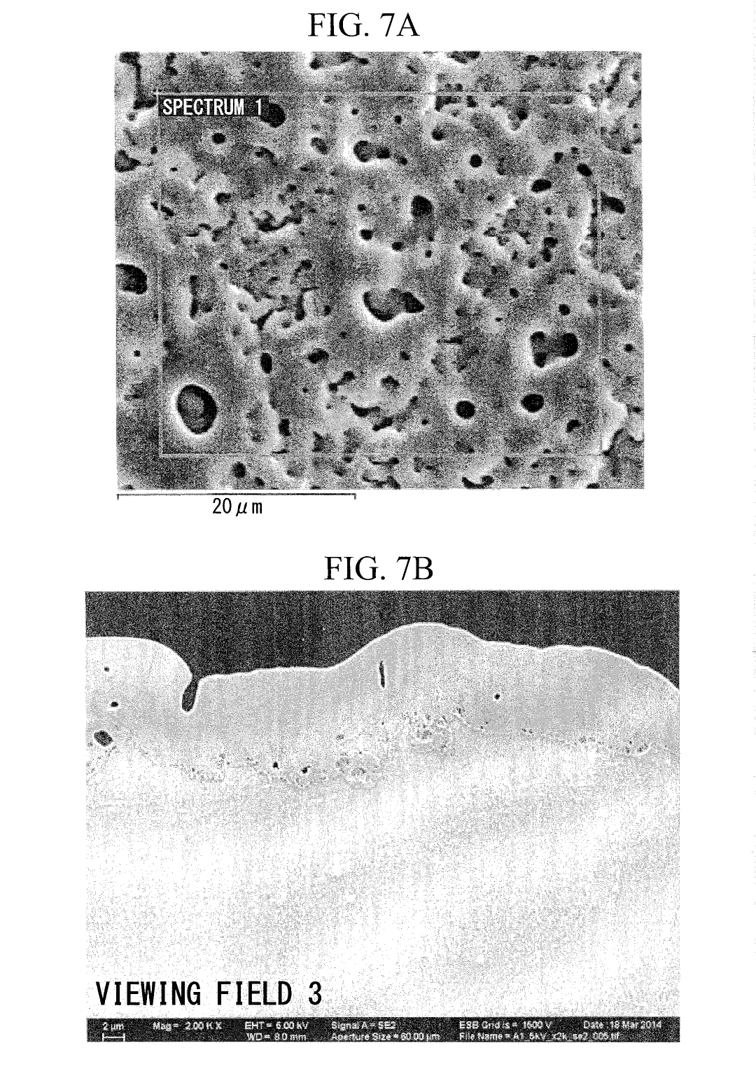

FIG. 6A shows an electron micrograph of an outer surface of an osteosynthetic implant 1 that is manufactured by means of a Second Example of the manufacturing method according to this embodiment, and FIG. 6B shows an electron micrograph of a tomographic image showing a portion from the anodic oxide coating 3 to the substrate 2.

In the Second Example, manufacturing is performed by setting the phosphoric-acid concentration to 0.05 mol/L, the current density at the surface of the substrate 2 to 30 A/dm², and the voltage of the applied voltage that is finally reached when stopping the passing of electricity to 350 V.

By doing so, the mass concentration of carbon atom at the outer surface of the anodic oxide coating 3 was 4.19%.

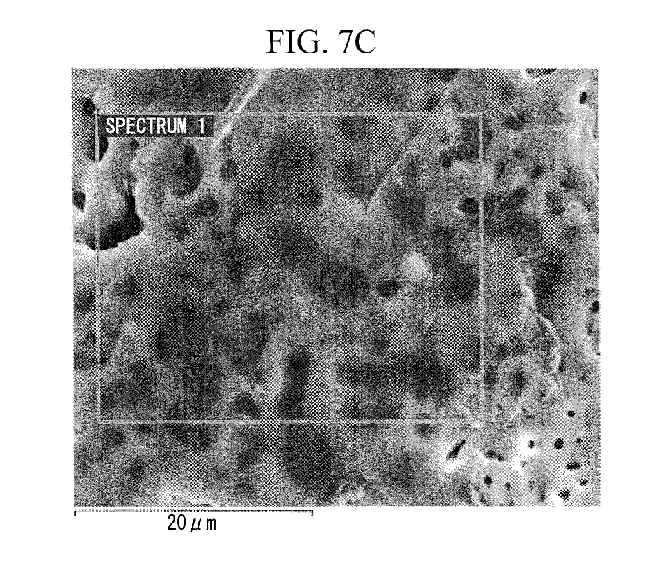

As a Comparative Example, FIG. 7A shows an electron micrograph of an outer surface of an anodic oxide coating 3 to which carbon is not adsorbed and that has a surface structure in which the Wenzel model is dominant, FIG. 7B shows an electron micrograph that shows a tomographic image showing a portion from anodic oxide coating 3 to the substrate 2 thereof, and FIG. 7C shows an electron micrograph of the outer surface thereof to which carbon is adsorbed. The mass concentration of carbon atom at the outer surface of the anodic oxide coating 3 in this case was 39.47%.

As a result, the following aspect is read from the above described embodiment of the present invention.

An aspect of the present invention is an osteosynthetic implant including: a substrate that is formed of magnesium or a magnesium alloy; and a porous anodic oxide coating that is formed on a surface of the substrate, wherein the anodic oxide coating has an outer surface that, due to the sizes and distribution of pores that are formed when generating the anodic oxide coating by means of anodic oxidation treatment, structurally prevents water from entering the pores while maintaining hydrophilicity thereof.

With this aspect, because the outer surface of the anodic oxide coating possesses hydrophilicity, the osteoconductivity is maintained, and, because the structure that prevents water from entering the pores is provided, generation of and contamination by carbide formed by bonding of water remaining in the pores and carbon atoms in the surrounding area are prevented, and thus, it is possible to prevent the osteoconductivity from being decreased. Because such characteristics are structurally imparted due to the sizes and the distribution of the pores formed when generating the anodic oxide coating by means of anodic oxidation treatment, it is not necessary to perform treatment to restore the hydrophilicity, such as sandblasting, etching, or the like, and thus, it is possible to achieve firm fusion with a bone after implanting due to the high osteoconductivity.

In the above-described aspect, the outer surface of the anodic oxide coating may have a surface structure in which the Cassie-Baxter model is dominant over the Wenzel model.

By doing so, when droplets are attached to the outer surface, a state in which a high hydrophilicity is achieved because the rough outer surface and the liquid surface appear to be in contact with each other over a large area due to a large degree of micro-scale irregularities caused by the distribution of the pores, whereas a state in which the droplets and the outer surface are in point contact with each other due to the presence of the numerous pores which the droplets cannot enter becomes dominant. By doing so, it is possible to enhance the osteoconductivity by preventing the droplets from entering the pores and by preventing carbide from remaining therein.

In the above-described aspect, at the outer surface of the anodic oxide coating, a ratio of areas of openings of the pores and areas of portions other than those may be equal to or less than 1.8.

By doing so, it is possible to make the Cassie-Baxter model dominant at the outer surface of the anodic oxide coating.

In the above-described aspect, at the outer surface of the anodic oxide coating, a ratio of areas of openings of the pores and areas of portions other than those may be equal to or less than 1.

By doing so, it is possible to more reliably prevent droplets from entering the pores.

In the above-described aspect, a coating thickness of the anodic oxide coating may be 1 to 5 μm, and an average pore size of the pores opened in the outer surface may be equal to or less than 5 μm.

In the above-described aspect, a coating thickness of the anodic oxide coating may be 1 to 5 μm, and an average pore size of the pores opened in the outer surface may be equal to or less than 1 μm. By doing so, it is possible to more reliably prevent droplets from entering the pores even if there is variability in the coating thickness.

By doing so, in the case in which the thickness of the anodic oxide coating is 1 to 5 μm, the Cassie-Baxter model and the Wenzel model coexist, which prevents moisture from remaining in the pores, and thus, it is possible to prevent contamination by carbide.

In the above-described aspect, a macro-scale surface roughness of the outer surface of the anodic oxide coating may be equal to or less than 1 μm.

By doing so, it is possible to decrease the apparent wettability of the outer surface of the anodic oxide coating.

In the above-described aspect, the anodic oxide coating may be formed by immersing the substrate formed of magnesium or a magnesium alloy in an electrolyte, which contains phosphoric acid at 0.1 mol/L or less, which contains ammonia or ammonium ion at 0.2 mol/L, which does not contain fluorine and chlorine, and which has a pH of 9-13, and electricity is passed therethrough.

Another aspect of the present invention is an osteosynthetic-implant manufacturing method in which anodic oxidation treatment is applied, in which a substrate formed of magnesium or a magnesium alloy is immersed in an electrolyte, which contains phosphoric acid at 0.1 mol/L or less, which contains ammonia or ammonium ion at 0.2 mol/L, which does not contain fluorine and chlorine, and which has a pH of 9-13, and electricity is passed therethrough.

### REFERENCE SIGNS LIST

1 osteosynthetic implant
2 substrate
3 anodic oxide coating
3a pore

1. An osteosynthetic implant comprising:
a substrate that is formed of magnesium or a magnesium alloy; and
a porous anodic oxide coating that is formed on a surface of the substrate,
wherein the anodic oxide coating has an outer surface that, due to the sizes and distribution of pores that are formed when generating the anodic oxide coating by means of anodic oxidation treatment, structurally prevents water from entering the pores while maintaining hydrophilicity thereof.

2. An osteosynthetic implant according to claim 1, wherein the outer surface of the anodic oxide coating has a surface structure in which the Cassie-Baxter model is dominant over the Wenzel model.

3. An osteosynthetic implant according to claim 2, wherein, at the outer surface of the anodic oxide coating, a ratio of areas of openings of the pores and areas of portions other than those is equal to or less than 1.8.

4. An osteosynthetic implant according to claim 3, wherein, at the outer surface of the anodic oxide coating, a ratio of areas of openings of the pores and areas of portions other than those is equal to or less than 1.

5. An osteosynthetic implant according to claim 1, wherein a coating thickness of the anodic oxide coating is 1 to 5 μm, and an average pore size of the pores opened in the outer surface is equal to or less than 5 μm.

6. A osteosynthetic implant according to claim 4, wherein a coating thickness of the anodic oxide coating is 1 to 5 μm, and an average pore size of the pores opened in the outer surface is equal to or less than 1 μm.

7. An osteosynthetic implant according to claim 1, wherein a macro-scale surface roughness of the outer surface of the anodic oxide coating is equal to or less than 1 μm.

8. An osteosynthetic implant according to claim 1, wherein the anodic oxide coating is formed by anodic oxidation treatment in which the substrate formed of magnesium or a magnesium alloy is immersed in an electrolyte, which contains phosphoric acid at 0.1 mol/L or less, which contains ammonia or ammonium ion at 0.2 mol/L, which does not contain fluorine and chlorine, and which has a pH of 9-13, and electricity is passed therethrough.

9. An osteosynthetic-implant manufacturing method in which anodic oxidation treatment is applied, in which a substrate formed of magnesium or a magnesium alloy is immersed in an electrolyte, which contains phosphoric acid at 0.1 mol/L or less, which contains ammonia or ammonium ion at 0.2 mol/L, which does not contain fluorine and chlorine, and which has a pH of 9-13, and electricity is passed therethrough.
